(19) United States
(12) Patent Application Publication
(10) Pub. No.: US 2018/0362493 A1
(43) Pub. Date: Dec. 20, 2018

(54) DINAPHTHOTHIOPHENE COMPOUNDS

(71) Applicant: Saint Louis University, St. Louis, MO (US)

(72) Inventors: Ryan D. McCulla, St. Louis, MO (US); John T. Petroff, II, St. Louis, MO (US); Satyanarayana M. Chintala, St. Louis, MO (US); John C. Throgmorton, St. Louis, MO (US)

(73) Assignee: Saint Louis University, St. Louis, MO (US)

(21) Appl. No.: 16/013,432

(22) Filed: Jun. 20, 2018

Related U.S. Application Data
(60) Provisional application No. 62/522,278, filed on Jun. 20, 2017.

(51) Int. Cl. C07D 333/50

(52) U.S. Cl. CPC C07D 333/50

(57) ABSTRACT

The present invention generally relates to various dinaphthothiophene compounds and processes for preparing these compounds.

### CROSS-REFERENCE TO RELATED APPLICATIONS

This application claims benefit of U.S. provisional application Ser. No. 62/522,278, filed Jun. 20, 2017, the entire disclosure of which is incorporated herein by reference.

### STATEMENT REGARDING FEDERALLY SPONSORED RESEARCH OR DEVELOPMENT

This invention was made with Government support under grant CHE-1255270 awarded by the National Science Foundation. The Government has certain rights in the invention.

### FIELD OF THE INVENTION

The present invention generally relates to various dinaphthothiophene compounds and processes for preparing these compounds.

### BACKGROUND OF THE INVENTION

Dinaphthothiophenes (DNTs) are a class of compounds with potential uses in organic semiconductors and the synthesis of asymmetric catalysts. Symmetrical or asymmetrical addition of functional groups to the dinaphthothiophene structure may be desired for steric bulk in binaphthyl catalyst synthesis or tuning the electronic properties of semiconductors or photooxygen precursors. Thus, versatility of functional group addition is a great asset in DNT synthesis. Until now, no versatile and concise methods for the synthesis of asymmetrically substituted dinaphthothiophenes have been reported.

Dinaphthothiophenes are a class of compounds structurally similar to thiophene-based organic semiconductors [1-3] and have shown promise for use in p-type organic semiconductors.[4,5] Dinaphthothiophenes have also been used as precursors to axially chiral 1,1′-binaphthyl catalysts, which play a large role in asymmetric synthesis. [6-8] In addition, dibenzothiophene S-oxide (DBTO) and its derivatives are a class of compounds suggested to release O(³P) upon irradiation with UV light.[9-12] Dinaphthothiophene S-oxides and other fused ring thiophene S-oxides have been investigated for their potential to release atomic oxygen during irradiation at longer wavelengths.[13] Despite their potential applications, few efficient ways to prepare asymmetrically substituted dinaphthothiophenes have been reported.

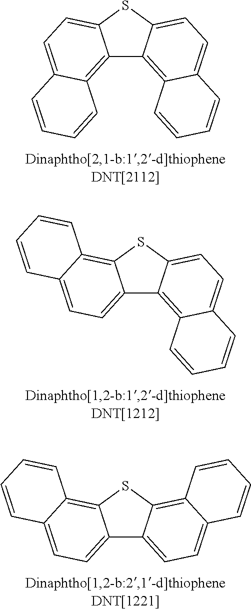 [SMILES: c1ccc2c(c1)ccc1sc3ccc4ccccc4c3c12 ; c1ccc2c(c1)ccc1c2sc2ccc3ccccc3c21 ; c1ccc2c(c1)ccc1c3ccc4ccccc4c3sc21]

Dinaphtho[2,1-b:1′,2′-d]thiophene “DNT-2112,” dinaphtho[1,2-b;1′,2′-d]thiophene “DNT-1212,” and dinaphtho[1,2-b:2′,1′-d]thiophene “DNT-1221” are three types of DNTs whose syntheses have previously been reported. DNT-2112 has been synthesized from the Newman-Kwart rearrangement of dithiocarbamates by heating the dimethylthiocarbamate of binaphthol neat at 285-310° C. to give the DNT-2112 in 20-40% yield [8,14,15], from dinaphthyl sulfide using an iodine-catalyzed photocyclization in 85% yield [16], and from cyclization of alkynes by heating ethynyl sulfides in benzene at 200° C. in a cascade cycloaromatization with 10% yield.[17] Rabindran and Tilak performed the condensation of 2-bromo-1-tetralone with 2-naphthalenethiol or 1-naphthalenethiol, followed by cyclization with P₂O₅ in phosphoric acid and dehydrogenation with selenium, giving DNT-2112 and DNT-1212 in 78% and 76% overall yield, respectively.[18] Morrison and Musgrave used the condensation of thiophene with 1,2-diphenylethanone to give (E,E)-2,5-bis(α-phenylstyryl)thiophene.[19] The phenyl-substituted distyrylthiophene was then photocyclized with iodine to give the diphenyl substituted DNT-2112 in 10% yield. The drawbacks of these preparations of DNT-2112 are that they give low overall yields and take two to four steps to prepare.

DNT-1221 derivatives have been made from the reaction of naphthalene-1-sulfonic acid dimethylamide with n-butyllithium and S₈ in 29-37% yield. [20] In addition, both DNT-2112 and DNT-1221 have been synthesized from dinaphthyl sulfides using a potassium tert-butoxide or n-butyllithium induced cyclodehydrogenation in 18-31% yield. [21,22] These syntheses of DNT-1221 suffer from low overall yields. While the final cyclization reactions to synthesize all three varieties of DNTs usually require only one step, anywhere from one to six steps may be required to synthesize the precursors needed for the cyclization reaction from commercially available materials. In addition, the methods requiring fewer steps to reach the cyclization precursor tend to have a more limited scope of synthesis. For example, the method of Morrison and Musgrave, which is the sole method to require only one step to achieve the cyclization precursor, lacks the ability to generate unsubstituted DNTs and has only been used to make diphenyl substituted DNT-2112.[19].

All three classes of DNTs have been synthesized from the flash vacuum pyrolysis of diethynyl/dichlorovinyl-diphenylthiophenes in 7-89% yield.[23] The flash pyrolysis method has some capability for functionalization of the DNT structure. However, it is only able to functionalize symmetrically, which limits the potential for the tuning of the electronic properties of DNTs by tuning functional groups. Furthermore, Tedjamulia et al. prepared all three classes of DNTs from formyl-benzonaphthothiophenes.[24] A Horner-Wadsworth-Emmons reaction was used to add a styrene unit, followed by a iodine-catalyzed photocyclization which gave yields of 45-76%. The synthetic route created here has potential for use in asymmetric DNT substitution, since the DNT core structure is assembled one half at a time; however, controlling the final position of the functional group would be difficult due to the variability of products of the final photocyclization step. This method also has the disadvantage of requiring three to six steps to reach the photocyclization precursor.

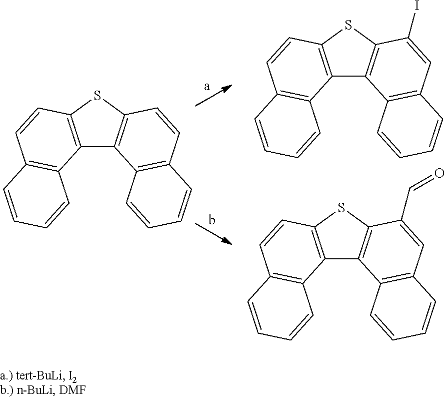

While DNTs have previously been asymmetrically functionalized, this has typically been done after the synthesis of the DNT structure. Cho et al. have taken DNT-2112, and functionalized the 6-position of DNT-2112 by use of tert-butyllithium and iodine.[8] In addition, n-butyllithium and DMF have been used to add an aldehyde group, also at the 6-position. [25,26] This indicates that the 6 (and 8) positions of DNT-2112 are selectively deprotonated by bases such as n-butyllithium, leaving the other positions on the dinaphthothiophene rings unable to be so similarly substituted.

In short, a variety of synthetic routes have previously been reported to produce unfunctionalized and a few functionalized DNTs. However, none of these methods begin with the thiophene ring and therefore require a greater number of steps to reach the dinaphthothiophene structure. Furthermore, these methods do not provide a simple way to asymmetrically incorporate functional groups onto the DNT structure.

### BRIEF SUMMARY OF THE INVENTION

The present invention is directed to various dinaphthothiophene compounds including compounds of Formula I (i.e., derivatives of DNT-1212):

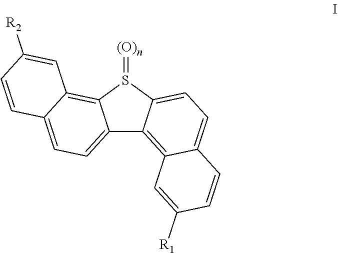

wherein R₁ is hydrogen, hydroxy, substituted or unsubstituted C₁-C₂₀ alkyl, or substituted or unsubstituted aryl; R₂ is hydrogen, hydroxy, substituted or unsubstituted C₁-C₂₀ alkyl, or substituted or unsubstituted aryl; and n is 0, 1, or 2.

Other dinaphthothiophene compounds of the present invention include compounds of Formula II (i.e., derivatives of DNT-2112):

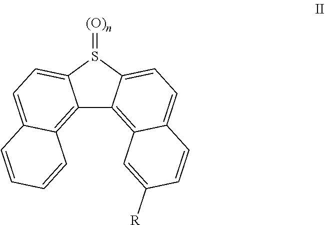

wherein R is hydrogen, hydroxy, substituted or unsubstituted C₁-C₂₀ alkyl, or substituted or unsubstituted aryl; and n is 0, 1, or 2.

Still further dinaphthothiophene compounds of the present invention include compounds of Formula III (i.e., derivatives of DNT-1221):

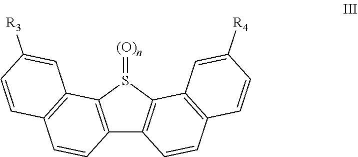

wherein R₃ is hydrogen, hydroxy, substituted or unsubstituted C₁-C₂₀ alkyl, or substituted or unsubstituted aryl; R₄ is hydrogen, hydroxy, substituted or unsubstituted C₁-C₂₀ alkyl, or substituted or unsubstituted aryl; and n is 0, 1, or 2.

The present invention also relates to various processes for preparing these compounds.

Other objects and features will be in part apparent and in part pointed out hereinafter.

### DETAILED DESCRIPTION OF THE INVENTION

The present invention generally relates to substituted derivatives of DNT-2112, DNT-1212, and DNT-1221 and processes to synthesize these compounds starting from mono- and dibromine-substituted thiophenes.

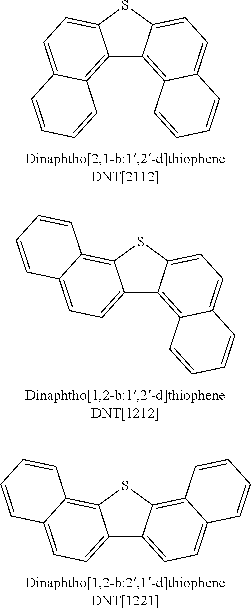 [SMILES: c1ccc2c(c1)ccc1sc3ccc4ccccc4c3c12 ; c1ccc2c(c1)ccc1c2sc2ccc3ccccc3c21 ; c1ccc2c(c1)ccc1c3ccc4ccccc4c3sc21]

Each of these routes involves only 3-4 steps in total, and contains the potential for the symmetric and asymmetric introduction of a wide variety of functional groups. Consequently, these processes can be used to synthesize a wide variety of functionalized dinaphthothiophenes.

The derivatives of three different classes of thiophenes (dinaphtho[2,1-b:1′,2′-d]thiophene, dinaphtho[1,2-b;1′,2′-d]thiophene, and dinaphtho[1,2-b:2′,1′-d]thiophene) were created by three different processes. Each route followed the same general strategy, beginning with two styrene groups being sequentially added to a mono- or dibrominated thiophene by either Suzuki coupling or Horner-Wadsworth-Emmons reaction. Once the thiophene was doubly substituted with styrene units, a photocyclization reaction with iodine as an oxidative catalyst was used to create the final dinaphthothiophene structure. In contrast to other synthetic routes, asymmetrically substituted functional groups have been incorporated into the synthesis of the DNT structure itself. Methoxy, trifluoromethyl, and methyl functional groups were used to create substituted DNTs. These three groups were chosen for their differences in electronegativity, which could be used to tune the electronic properties of DNTs. Whereas only symmetrically-substituted DNTs had been synthesized before, now a wide variety of asymmetrically-substituted DNTs can be easily made and tuned for use in organic semiconductors and chiral 1,1′-binaphthyl catalysts.

Various dinaphthothiophene compounds of the present invention include compounds of Formula I (i.e., derivatives of DNT-1212):

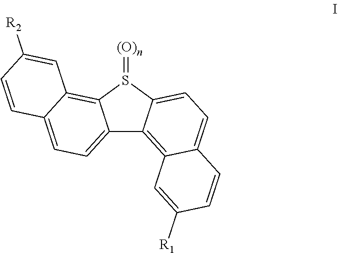

wherein R₁ is hydrogen, hydroxy, substituted or unsubstituted C₁-C₂₀ alkyl, or substituted or unsubstituted aryl; R₂ is hydrogen, hydroxy, or substituted or unsubstituted C₁-C₂₀ alkyl; and n is 0, 1, or 2.

In various embodiments, R₁ is hydrogen, hydroxy, C₁-C₂₀ alkyl, C₁-C₂₀ alkoxy, C₁-C₂₀ haloalkyl, C₁-C₂₀ haloalkoxy, aryl, alkyl-substituted aryl, halo-substituted aryl, or hydroxy-substituted aryl. In some embodiments, R₁ is hydrogen, hydroxy, C₁-C₁₀ alkyl, C₁-C₁₀ alkoxy, C₁-C₁₀ haloalkyl, C₁-C₁₀ haloalkoxy, aryl, alkyl-substituted aryl, halo-substituted aryl, or hydroxy-substituted aryl. In certain embodiments, R₁ is hydrogen, hydroxy, methyl, ethyl, propyl, butyl, methoxy, ethoxy, propoxy, trifluoromethyl, phenyl, hydroxyphenyl, ethylphenyl, carboxyphenyl, naphthyl, anthracenyl, biphenyl, tolyl, cumyl, styryl, ortho-xylyl, meta-xylyl, para-xylyl, fluorophenyl, chlorophenyl, bromobenzyl, or iodobenzyl.

In various embodiments, R₂ is hydrogen, hydroxy, C₁-C₂₀ alkyl, C₁-C₂₀ alkoxy, C₁-C₂₀ haloalkyl, C₁-C₂₀ haloalkoxy, aryl, alkyl-substituted aryl, halo-substituted aryl, or hydroxy-substituted aryl. In some embodiments, R₂ is hydrogen, hydroxy, C₁-C₁₀ alkyl, C₁-C₁₀ alkoxy, C₁-C₁₀ haloalkyl, C₁-C₁₀ haloalkoxy, aryl, alkyl-substituted aryl, halo-substituted aryl, or hydroxy-substituted aryl. In certain embodiments, R₂ is hydrogen, hydroxy, methyl, ethyl, propyl, butyl, methoxy, ethoxy, propoxy, trifluoromethyl, phenyl, hydroxyphenyl, ethylphenyl, carboxyphenyl, naphthyl, anthracenyl, biphenyl, tolyl, cumyl, styryl, ortho-xylyl, meta-xylyl, para-xylyl, fluorophenyl, chlorophenyl, bromobenzyl, or iodobenzyl.

In some embodiments, R₁ and R₂ are different.

Various processes of the present invention are directed to preparing the dinaphthothiophene compounds of Formula I. In general, these processes comprise one of more of the following steps of:

reacting, in the presence of a catalyst comprising palladium, a base, and organic solvent, 2,4-dibromothiophene with a phenylethenyl boronic acid compound of Formula A-1:

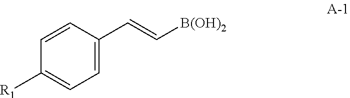

to form a 4-bromo-2-styrylthiophene compound of Formula A-2:

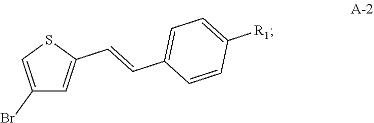

reacting, in the presence of a catalyst comprising palladium, a base, and organic solvent, the 4-bromo-2-styrylthiophene compound of Formula A-2 with a phenylethenyl boronic acid compound of Formula A-3:

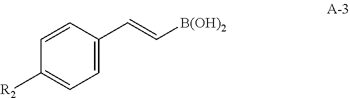

to form a 2,4-distryrylthiophene compound of Formula A-4:

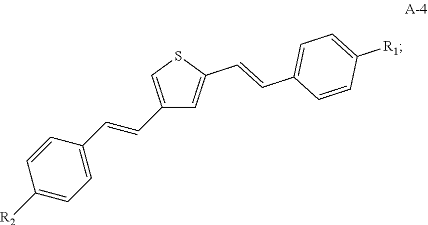

irradiating the compound of Formula A-4 with UV light in the presence of iodine and propylene oxide to form a dinaphthothiophene compound of Formula I-A:

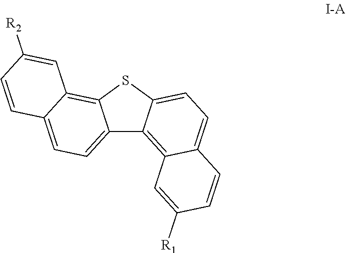

wherein R₁, and R₂ are as defined above for Formula I. A further oxidation step can be performed to yield the compounds of Formula I-B:

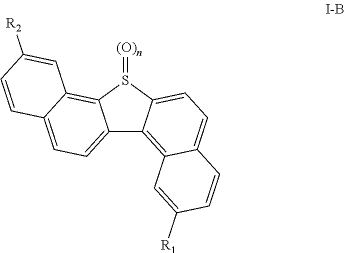

where R₁, and R₂ are as defined above for Formula I and n is 1 or 2. The oxidation can be performed using an oxidant such as meta-chloroperoxybenzoic acid.

Other dinaphthothiophene compounds of the present invention include compounds of Formula II (i.e., derivatives of DNT-2112):

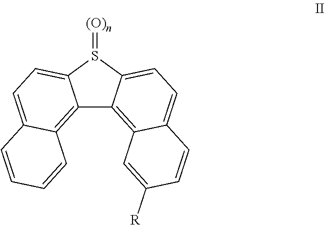

wherein R is hydrogen, hydroxy, substituted or unsubstituted C₁-C₂₀ alkyl, or substituted or unsubstituted aryl; and n is 0, 1, or 2.

In various embodiments, R is hydrogen, hydroxy, C₁-C₂₀ alkyl, C₁-C₂₀ alkoxy, C₁-C₂₀ haloalkyl, C₁-C₂₀ haloalkoxy, aryl, alkyl-substituted aryl, halo-substituted aryl, or hydroxy-substituted aryl. In some embodiments, R is hydrogen, hydroxy, C₁-C₁₀ alkyl, C₁-C₁₀ alkoxy, C₁-C₁₀ haloalkyl, C₁-C₁₀ haloalkoxy, aryl, alkyl-substituted aryl, halo-substituted aryl, or hydroxy-substituted aryl. In certain embodiments, R is hydrogen, methyl, ethyl, propyl, butyl, methoxy, ethoxy, propoxy, or trifluoromethyl. In some embodiments, R is hydrogen, hydroxy, methyl, ethyl, propyl, butyl, methoxy, ethoxy, propoxy, trifluoromethyl, phenyl, hydroxyphenyl, ethylphenyl, carboxyphenyl, naphthyl, anthracenyl, biphenyl, tolyl, cumyl, styryl, ortho-xylyl, meta-xylyl, para-xylyl, fluorophenyl, chlorophenyl, bromobenzyl, or iodobenzyl.

Further processes of the present invention are directed to preparing the dinaphthothiophene compounds of Formula II. In general, these processes comprise the steps of:

reacting 2-bromthiophene with trans-2-(4-phenyl)vinylboronic acid in the presence of a catalyst comprising palladium, a base, and organic solvent to form (E)-2-styrylthiophene;

formylating (E)-2-styrylthiophene to form (E)-5-styrylthiophene-2-carbaldehyde;

reacting, in the presence of sodium hydride, (E)-5-styrylthiophene-2-carbaldehyde with a 4-substituted phosphonic acid diethyl ester compound of Formula B-1:

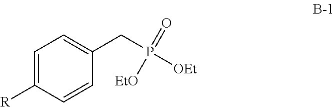

to form a compound of Formula B-2:

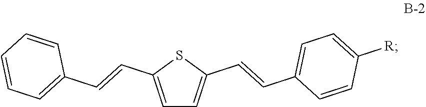

irradiating the compound of Formula B-2 with UV light in the presence of iodine and propylene oxide to form a dinaphthothiophene compound of Formula II-A:

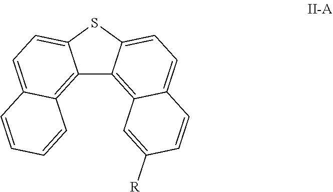

wherein R is as defined for Formula II. A further oxidation step can be performed to yield the compounds of Formula II-B:

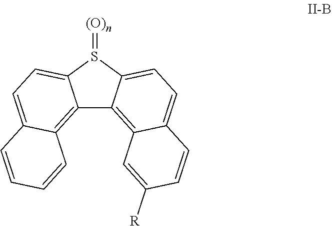

wherein R is as defined for Formula II and n is 1 or 2. The oxidation can be performed using an oxidant such as meta-chloroperoxybenzoic acid.

Still further dinaphthothiophene compounds of the present invention include compounds of Formula III (i.e., derivatives of DNT-1221):

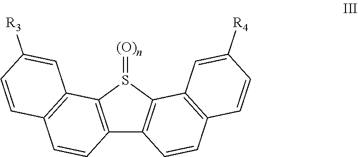

wherein R₃ is hydrogen, hydroxy, substituted or unsubstituted C₁-C₂₀ alkyl, or substituted or unsubstituted aryl and R₄ is hydrogen, hydroxy, substituted or unsubstituted C₁-C₂₀ alkyl, or substituted or unsubstituted aryl; and n is 0, 1, or 2.

In various embodiments, R₃ is hydrogen, hydroxy, C₁-C₂₀ alkyl, C₁-C₂₀ alkoxy, C₁-C₂₀ haloalkyl, C₁-C₂₀ haloalkoxy, aryl, alkyl-substituted aryl, halo-substituted aryl, or hydroxy-substituted aryl. In some embodiments, R₃ is hydrogen, hydroxy, C₁-C₁₀ alkyl, C₁-C₁₀ alkoxy, C₁-C₁₀ haloalkyl, C₁-C₁₀ haloalkoxy, aryl, alkyl-substituted aryl, halo-substituted aryl, or hydroxy-substituted aryl. In certain embodiments, R₃ is hydrogen, hydroxy, methyl, ethyl, propyl, butyl, methoxy, ethoxy, propoxy, trifluoromethyl, phenyl, hydroxyphenyl, ethylphenyl, carboxyphenyl, naphthyl, anthracenyl, biphenyl, tolyl, cumyl, styryl, ortho-xylyl, meta-xylyl, para-xylyl, fluorophenyl, chlorophenyl, bromobenzyl, or iodobenzyl.

In various embodiments, R₄ is hydrogen, hydroxy, C₁-C₂₀ alkyl, C₁-C₂₀ alkoxy, C₁-C₂₀ haloalkyl, C₁-C₂₀ haloalkoxy, aryl, alkyl-substituted aryl, halo-substituted aryl, or hydroxy-substituted aryl. In some embodiments, R₄ is hydrogen, hydroxy, C₁-C₁₀ alkyl, C₁-C₁₀ alkoxy, C₁-C₁₀ haloalkyl, C₁-C₁₀ haloalkoxy, aryl, alkyl-substituted aryl, halo-substituted aryl, or hydroxy-substituted aryl. In certain embodiments, R₄ is hydrogen, hydroxy, methyl, ethyl, propyl, butyl, methoxy, ethoxy, propoxy, trifluoromethyl, phenyl, hydroxyphenyl, ethylphenyl, carboxyphenyl, naphthyl, anthracenyl, biphenyl, tolyl, cumyl, styryl, ortho-xylyl, meta-xylyl, para-xylyl, fluorophenyl, chlorophenyl, bromobenzyl, or iodobenzyl.

In various embodiments, R₃ and R₄ are different.

Further processes of the present invention are directed to preparing the dinaphthothiophene compounds of Formula III. In general, these processes comprise the steps of:

formylating 3,4-dibromothiophene to form 4-bromothiophene-3-carbaldehyde;

reacting, in the presence of a catalyst comprising palladium, a base, and organic solvent, 4-bromothiophene-3-carbaldehyde with a phenylethenyl boronic compound of Formula C-1:

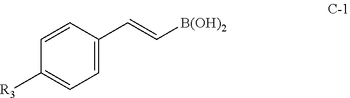

to form a 3-formyl-4-styrylthiophene compound of Formula C-2:

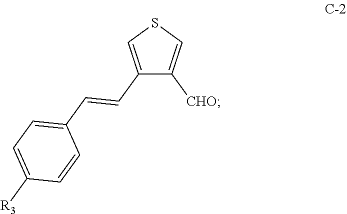

reacting, in the presence of sodium hydride, the 3-formyl-4-styrylthiophene compound of Formula C-2 with a 4-substituted phosphonic acid diethyl ester compound of Formula C-3:

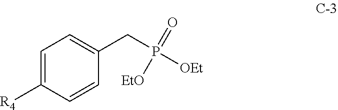

to form a compound of Formula C-4:

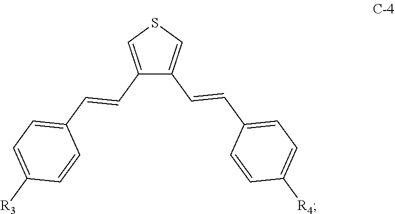

irradiating the compound of Formula C-4 with UV light in the presence of iodine and propylene oxide to form the dinaphthothiophene compound of Formula III-A:

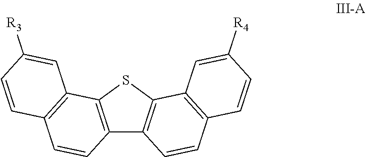

wherein R₃ and R₄ are as defined for Formula III above. A further oxidation step can be performed to yield the compounds of Formula III-B:

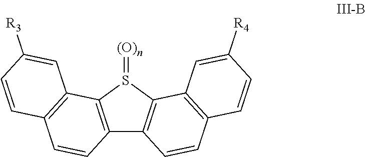

wherein R₃ and R₄ are as defined for Formula III above and n is 1 or 2. The oxidation can be performed using an oxidant such as meta-chloroperoxybenzoic acid.

Unless otherwise indicated, the alkyl groups described herein are preferably lower alkyl groups containing from 1 to 20 carbon atoms in the principal chain. They may be straight or branched chain or cyclic. Also, unless otherwise indicated, the substituted alkyl groups described herein can contain saturated or unsaturated and branched or unbranched carbon chains having from 1 to 20 carbon atoms in the principal chain.

### Examples

The following non-limiting examples are provided to further illustrate the present invention.

### Example 1. Synthesis of Dinaphtho[1,2-b;1′,2′-d]thiophenes

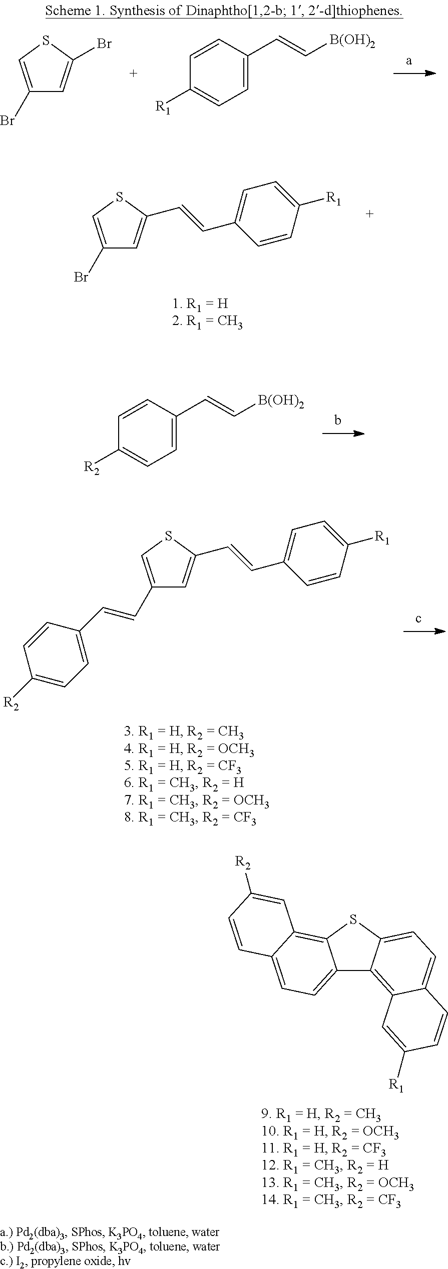

DNT-1212 derivatives were synthesized using the path shown in Scheme 1. This synthetic path used for the synthesis of asymmetrically substituted DNT-1212 derivatives began with 2,4-dibromothiophene. The bromine in the 2-position is preferentially substituted over the 4-position in carbon-carbon coupling reactions to give an asymmetric product.[27-32]. Therefore, a Suzuki-Miyaura reaction with one equivalent of [(E)-2-phenylethenyl]boronic acid or [(E)-2-[4-(methyl)phenyl]ethenyl]boronic acid was performed to add the first styrene unit to give 4-bromo-2-styrylthiophenes 1 and 2 (Table 1). A second Suzuki-Miyaura reaction was used to add a second styryl group in the 4-position. This second coupling was successful with 4-substituted styrylboronic acids to give 2,4-distyrylthiophenes 3-8 (Table 1). These Suzuki-Miyaura couplings gave yields anywhere from 10% to 81% depending on the substituent on the boronic acid. Reactions were performed at temperatures ranging from 55° C. to 95° C.; however, no significant change in yield was noticed. Unsubstituted styrylboronic acids gave the highest yields, followed by trifluoromethyl-substituted styrylboronic acids. Methoxy-substituted boronic acids gave the lowest yields. In the next step of this synthetic route, 2,4-distyrylthiophenes 3-8 were irradiated with UVC light in the presence of iodine and propylene oxide to fuse the rings, giving DNTs 9-14 (Table 2). This photoreaction proceeds by an oxidative mechanism: first, a photoinduced electrocyclization to create the C—C bond, followed by an oxidative dehydrogenation catalyzed by iodine to regain aromaticity.[33] The photocyclization of 2,4-distyrylthiophenes gave yields of 20-31%, with the exception of 4. The photocyclization resulting in compound 4 gave a higher yield of 66%, in contrast to most other reactions performed involving methoxy-substituted reactants, which had significantly lower yields on average than those involving different substituents.

TABLE 1
Suzuki-Miyaura Reactions.

| | | | | | Water | |
|---|---|---|---|---|---|---|
| | Bromo- | | Equiv. | Temp. | (% of | Yield |
| Entry | thiophene | Product | R—B(OH)₂ | (° C.) | solvent) | (%) |
| 1 | 2,4-dibromo- | 1 | 1.1 | 95 | 11 | 81 |
| | thiophene | | | | | |
| 2 | 2,4-dibromo- | 2 | 1.1 | 90 | 20 | 29 |
| | thiophene | | | | | |
| 3 | 1 | 3 | 1.2 | 80 | 17 | 46 |
| 4 | 1 | 4 | 1.1 | 85 | 17 | 10 |
| 5 | 1 | 5 | 1.2 | 55 | 17 | 48 |
| 6 | 2 | 6 | 1.2 | 80 | 20 | 36 |
| 7 | 2 | 7 | 1.1 | 75 | 17 | 30 |
| 8 | 2 | 8 | 1.1 | 75 | 17 | 31 |
| 9 | 2-Bromo- | 15 | 1.2 | 80 | 11 | 78 |
| | thiophene | | | | | |

TABLE 2
2,4-Distyrylthiophene Cyclization.

| Entry | Distyrylthiophene | Product | Time (days) | Yield (%) |
|---|---|---|---|---|
| 1 | 3 | 9 | 0.8 | 24 |
| 2 | 4 | 10 | 0.6 | 66 |
| 3 | 5 | 11 | 1.9 | 24 |
| 4 | 6 | 12 | 0.8 | 31 |
| 5 | 7 | 13 | 0.8 | 20 |
| 6 | 8 | 14 | 0.7 | 28 |

The 4-substituted trans-2-(phenylethenyl)boronic acids used in the Suzuki-Miyaura coupling step often underwent self-coupling rather than coupling with the bromothiophene. This created a side product which was detected by GCMS, with an m/z dependent on the boronic acid used. For example, Suzuki reactions involving unsubstituted styrylboronic acids in Table 1 (Entries 1, 3, and 6), gave a product with a m/z of 206 and was believed to be 1,4-diphenyl-1,3-butadiene. The purification of the desired products was complicated by the presence of these self-coupled byproducts, especially in the case of 7. Propylene oxide was added to all photocyclizations to quench the HI resulting from the reaction. In every photoreaction, care was taken not to irradiate the solution past completion, which would result in both a decreased yield and a white precipitate that was insoluble in organic solvents. In the absence of iodine, most photoreactions still occurred; however, they proceeded more slowly. In the synthesis of both DNT-1221 derivatives and DNT-2112 derivatives, reactions involving a methoxy substituent gave lower yields than those involving other substituents.

### Example 2. Synthesis of Dinaphtho[1,2-b;1′,2′-d]thiophenes

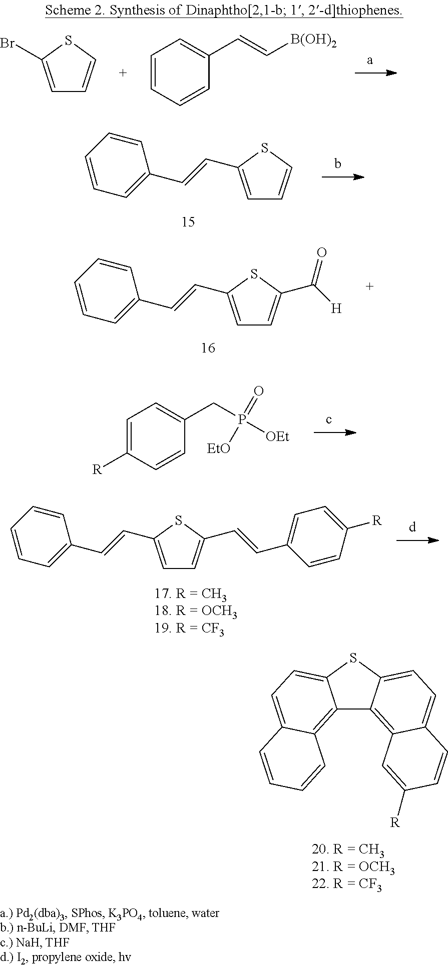

DNT-2112 derivatives 20-22 were created using the synthetic route shown in Scheme 2. First, 2-bromothiophene was coupled with a trans-2-(4-Phenyl)vinylboronic acid using a Suzuki-Miyaura coupling to create compound 15 in 76% yield. [34] The 5-position of the thiophene ring was then formylated using n-Butyllithium and DMF, giving compound 16 in 36% yield. [35,36] The 5-position is preferentially formylated due to its relatively low pKₐ (˜33) compared to the 3 or 4 positions (˜39) resulting from its location next to the sulfur in the thiophene ring.[37] A Horner-Wadsworth-Emmons reaction using a 4-substituted phosphonic acid diethyl ester was used to add a second styryl group to the other side of the thiophene ring (Table 3) to create 2,5-distyrylthiophenes 17-19. The methoxy-substituted phosphonic acid diethyl ester gave a 14% yield that was significantly lower than the methyl and trifluoromethyl-substituted phosphonic esters, which gave yields of 58% and 76%, respectively. The DNTs 20-22 were created via the same oxidative photocyclization used in the synthesis of DNTs 9-14 (Table 4).

TABLE 3
Horner-Wadsworth-Emmons Reaction
with 2-Formyl-5-Styrylthiophene.

| | | | | | | |
|---|---|---|---|---|---|---|
| | | | | Phosphonic | Equiv. | |
| Entry | Aldehyde | Product | Equiv. | Ester | NaH | Yield (%) |

| | | | | | |
|---|---|---|---|---|---|
| 1 | 16 | 17 | 1.3 | 5.5 | 58 |
| 2 | 16 | 18 | 1.9 | 3.3 | 14 |
| 3 | 16 | 19 | 1.2 | 4.7 | 76 |

TABLE 4
2,5-Distyrylthiophene Cyclization.

| Entry | Distyrylthiophene | Product | Time (days) | Yield (%) |
|---|---|---|---|---|
| 1 | 17 | 20 | 6 | 1 |
| 2 | 18 | 21 | 27 | <1% |
| 3 | 19 | 22 | 1.5 | 29 |

The Horner-Wadsworth-Emmons reaction of 16 to yield 2,5-distyrylthiophenes 17-19 gave unreliable yields. Different variables, such as molar equivalents of sodium hydride and temperature during reagent addition, were changed with no consistent improvement in yield. The Horner-Wadsworth-Emmons reaction to give 18 (14% yield) and the subsequent photocyclization to give 21 (<1% yield) gave significantly lower yields than the reactions to produce 17 and 19. The photocyclization of 18 did not yield enough 21 to completely characterize, though it was detected by GCMS. This follows the trend seen in Table 1 (Entries 4 and 7) and Table 2 (Entry 5) where the Suzuki coupling and photocyclization of reactants containing the methoxy group gave lower yields. In addition, 2,5-distyrylthiophenes gave lower photocyclization yields than other distyrylthiophenes. Since DNT-2112 is known to adopt a twisted conformation, these lower yields are likely due to sterics hindering the cyclization of 2,5-distyrylthiophene into the planar shape of other fused thiophene structures.[13]

### Example 3. Synthesis of Dinaphtho[1,2-b;1′,2′-d]thiophenes

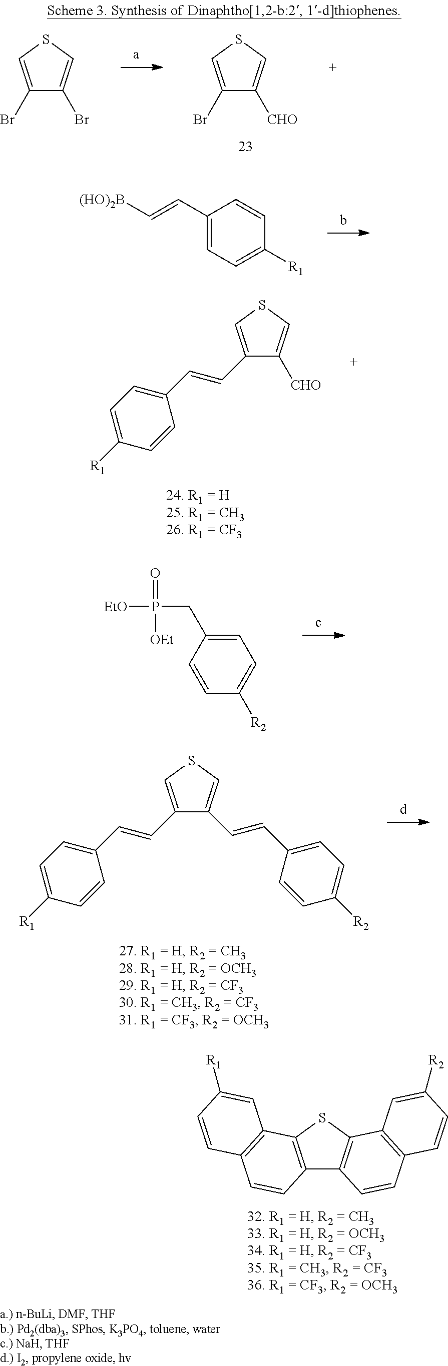

DNT-1221 derivatives 32-36 were synthesized using the route shown in Scheme 3. Formylation with n-butyllithium and DMF was used to convert 3,4-dibromothiophene to 3-bromothiophene-4-carbaldehyde 23 in 77% yield.[38,39] Suzuki-Miyaura coupling was then used to add the first styryl group to one side of the thiophene ring to give 3-formyl-4-styrylthiophenes 24-26 in 10-77% yield (Table 5), from which asymmetric distyrylthiophenes could easily be synthesized. The trifluoromethyl-substituted styrylboronic acid gave the highest yields. A Horner-Wadsworth-Emmons reaction was used to add the second substituted styryl group to the other side of the thiophene ring (Table 6), creating 3,4-distyrylthiophenes 27-31 in yields from 16-95%. The CF₃-substituted benzylphosphonic esters used in the creation of 29 and 30 gave higher yields compared to those with other substituents. The Suzuki-Miyaura coupling was performed before the Horner-Wadsworth-Emmons reaction in this route because the 1,4-diphenyl-1,3-butadiene byproducts formed from the Suzuki-Miyaura reaction were easier to separate from the more polar 3-formyl-4-styrylthiophenes than from 3,4-distyrylthiophenes. An oxidative photocyclization was then used in the same manner as the previous routes to fuse the rings together to give DNT-1221 derivatives 32-36 in yields of 6-20% (Table 7).

TABLE 5
Suzuki-Miyaura Reaction with 3-Bromo-4-Formylthiophene

| | | | Equiv. | Temp. | |
|---|---|---|---|---|---|
| Entry | Bromothiophene | Product | R-B(OH)₂ | (° C.) | Yield (%) |
| 1 | 23 | 24 | 1.2 | 95 | 77 |
| 2 | 23 | 25 | 1.5 | 70 | 46 |
| 3 | 23 | 26 | 1.2 | 86 | 86 |

TABLE 6
Horner-Wadsworth-Emmons Reaction
with 3-Formyl-4-Styrylthiophene

| Entry | Aldehyde | Product | Equiv. Phosphonic Ester | Yield (%) |
|---|---|---|---|---|
| 1 | 24 | 27 | 1.5 | 16 |
| 2 | 24 | 28 | 1.2 | 21 |
| 3 | 24 | 29 | 1.5 | 95 |
| 4 | 25 | 30 | 1.5 | 32 |
| 5 | 26 | 31 | 1.0 | 40 |

TABLE 7
3,4-Distyrylthiophene Cyclization

| Entry | Distyrylthiophene | Product | Time (hours) | Yield (%) |
|---|---|---|---|---|
| 1 | 27 | 32 | 8 | 7 |
| 2 | 28 | 33 | 6.5 | 20 |
| 3 | 29 | 34 | 7.5 | 18 |
| 4 | 30 | 35 | 5 | 9 |
| 5 | 31 | 36 | 3.5 | 6 |

A side product of the photoreaction of 27-31 is suspected to form by the ring closure of the thiophene ring as shown in Scheme 4, forming side products 37-41. The side product for Table 7, Entry 1 (37) was analyzed by GCMS and shown to have an m/z of 300, corresponding to the loss of H₂ by the oxidative dehydrocyclization mechanism. Solvents such as toluene, hexanes, and a mixture of dichloromethane and hexanes were tried in the photocyclization reaction, but there was no significant change in the product ratios. Preparative TLC was used to separate the photocyclization reaction products.

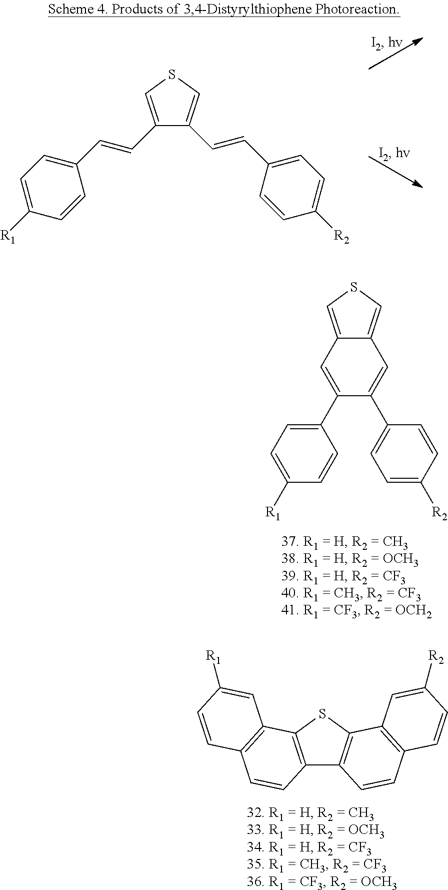

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions and processes without departing from the scope of the invention, it is intended that all matter contained in the above description shall be interpreted as illustrative and not in a limiting sense.

Having described the invention in detail, it will be apparent that modifications and variations are possible without departing from the scope of the invention defined in the appended claims.

### REFERENCES

[1] Xia, D.; Marszalek, T.; Li, M.; Guo, X.; Baumgarten, M.; Pisula, W.; Mullen, K. Org Lett 2015, 17, 3074.
[2] Feng, G.; Xu, Y.; Zhang, J.; Wang, Z.; Zhou, Y.; Li, Y.; Wei, Z.; Li, C.; Li, W. J Mater Chem A 2016, 4, 6056.
[3] Gupta, R. K.; Pradhan, B.; Pathak, S. K.; Gupta, M.; Pal, S. K.; Sudhakar, A. A. Langmuir 2015, 31, 8092.
[4] Nakahara, K.; Mitsui, C.; Okamoto, T.; Yamagishi, M.; Soeda, J.; Miwa, K.; Sato, H.; Yamano, A.; Uemura, T.; Takeya, J. Jpn J Appl Phys 2013, 52, 1.
[5] Okamoto, T.; Mitsui, C.; Yamagishi, M.; Nakahara, K.; Soeda, J.; Hirose, Y.; Miwa, K.; Sato, H.; Yamano, A.; Matsushita, T.; Uemura, T.; Takeya, J. Adv Mater 2013, 25, 6392.
[6] Cossu, S.; De Lucchi, O.; Fabbri, D.; Valle, G.; Painter, G. F.; Smith, R. A. J. Tetrahedron 1997, 53, 6073.
[7] Shimada, T.; Cho, Y.-H.; Hayashi, T. J Am Chem Soc 2002, 124, 13396.
[8] Cho, Y.-H.; Kina, A.; Shimada, T.; Hayashi, T. J Org Chem 2004, 69, 3811.
[9] Gurria, G. M.; Posner, G. H. J Org Chem 1973, 38, 2419.
[10] Gregory, D. D.; Wan, Z.; Jenks, W. S. J Am Chem Soc 1997, 119, 94.
[11] Thomas, K. B.; Greer, A. J Org Chem 2003, 68, 1886.
[12] Nag, M.; Jenks, W. S. J Org Chem 2005, 70, 3458.
[13] Zheng, X.; Baumann, S. M.; Chintala, S. M.; Galloway, K. D.; Slaughter, J. B.; McCulla, R. D. Photochem Photobiol Sci 2016, 15, 791.
[14] Bandarage, U. K.; Simpson, J.; Smith, R. A. J.; Weavers, R. T. Tetrahedron 1994, 50, 3463.
[15] Fabbri, D.; Delogu, G.; De Lucchi, 0. J Org Chem 1993, 58, 1748.
[16] Sadorn, K.; Sinananwanich, W.; Areephong, J.; Nerungsi, C.; Wongma, C.; Pakawatchai, C.; Thongpanchang, T. Tetrahedron Lett 2008, 49, 4519.
[17] Lewis, K. D.; Rowe, M. P.; Matzger, A. J. Tetrahedron 2004, 60, 7191.
[18] Rabindran, K.; Tilak, B. D. Proc Indian Acad Sci 1953, 38A, 271.
[19] Morrison, B. J.; Musgrave, O. C. J Chem Soc Perkin Trans 1 2002, 11, 1944.
[20] Alam, A.; Ohta, H.; Yamamoto, T.; Ogawa, S.; Sato, R. Heteroat Chem 2007, 18, 239.
[21] Ohgaki, H.; Mitsuhashi, H.; Suzuki, H. J Chem Res Synopses 2003, 5, 264.
[22] Vasu, D.; Yorimitsu, H.; Osuka, A. Angew Chemie Int Ed 2015, 54, 7162.
[23] Imamura, K.; Hirayama, D.; Yoshimura, H.; Takimiya, K.; Aso, Y.; Otsubo, T. Tetrahedron Lett 1999, 40, 2789.
[24] Tedjamulia, M. L.; Tominaga, Y.; Castle, R. N. J Heterocycl Chem 1983, 20, 1143.
[25] Nanbu, Y.; Nishikubo, T. Method of Increasing or Adjusting Refractive Index-Improving Effect by Dibenzothiophene Skeleton-Bearing Compound, 2011.
[26] Nanbu, Y.; Nishikubo, T. Refractive Index Improver, and Resin Composition, Polymerizable or Curable Composition, and Optical Material Including the Same, 2011.
[27] Rasheed, T.; Rasool, N.; Noreen, M.; Gull, Y.; Zubair, M.; Ullah, A.; Rana, U. A. J Sulfur Chem 2015, 36, 240.
[28] Chen, L.; Mali, K. S.; Puniredd, S. R.; Baumgarten, M.; Parvez, K.; Pisula, W.; De Feyter, S.; Müllen, K. J Am Chem Soc 2013, 135, 13531.
[29] Bey, E.; Marchais-Oberwinkler, S.; Werth, R.; Negri, M.; Al-Soud, Y. A.; Kruchten, P.; Oster, A.; Frotscher, M.; Birk, B.; Hartmann, R. W. J Med Chem 2008, 51, 6725.
[30] Schmitt, C.; Kail, D.; Mariano, M.; Empting, M.; Weber, N.; Paul, T.; Hartmann, R. W.; Engel, M. PLoS One 2014, 9, e87851.
[31] Gronowitz, S.; Svensson, A. Isr J Chem 1986, 27, 25.
[32] Proutiere, F.; Aufiero, M.; Schoenebeck, F. J Am Chem Soc 2012, 134, 606.
[33] De, P. K.; Neckers, D. C. Org Lett 2012, 14, 78.
[34] Ma, B.; Fiordeliso, J.; Wu, Y.; Kwong, R. Triphenylene-Benzofuran/Benzothiophene/Benzoselenophene Compounds with Substituents Joining to Form Fused Rings, 2011.
[35] Zhang, M.; Xu, H.; Peng, C.; Huang, H.; Bo, S.; Liu, J.; Liu, X.; Zhen, Z.; Qiu, L. RSC Adv 2014, 4, 15870.
[36] Martin-Santamaria, S.; Rodriguez, J.-J.; de Pascual-Teresa, S.; Gordon, S.; Bengtsson, M.; Garrido-Laguna, I.; Rubio-Viqueira, B.; Lopez-Casas, P. P.; Hidalgo, M.; de Pascual-Teresa, B.; Ramos, A. Org Biomol Chem 2008, 6, 3486.
[37] Shen, K.; Fu, Y.; Li, J.-N.; Liu, L.; Guo, Q.-X. Tetrahedron 2007, 63, 1568.
[38] Park, J. H.; Seo, Y. G.; Yoon, D. H.; Lee, Y.-S.; Lee, S.-H.; Pyo, M.; Zong, K. Eur Polym J 2010, 46, 1790.
[39] Zhu, D.; Sun, L.; Liu, Q.; Wen, S.; Han, L.; Bao, X.; Yang, R. Macromol Rapid Commun 2015, 36, 2065.

1. A process for preparing a dinaphthothiophene compound, the process comprising:
reacting, in the presence of a catalyst comprising palladium, a base, and organic solvent, 2,4-dibromothiophene with a phenylethenyl boronic acid compound of Formula A-1: 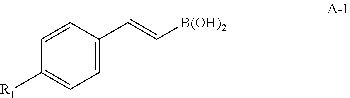
to form a 4-bromo-2-styrylthiophene compound of Formula A-2: 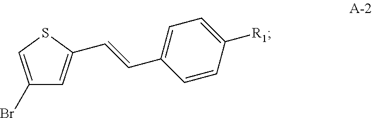
reacting, in the presence of a catalyst comprising palladium, a base, and organic solvent, the 4-bromo-2-styrylthiophene compound of Formula A-2 with a phenylethenyl boronic acid compound of Formula A-3: 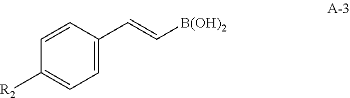
to form a 2,4-distryrylthiophene compound of Formula A-4: 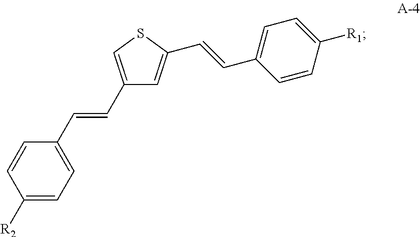
irradiating the compound of Formula A-4 with UV light in the presence of iodine and propylene oxide to form a dinaphthothiophene compound of Formula I-A: 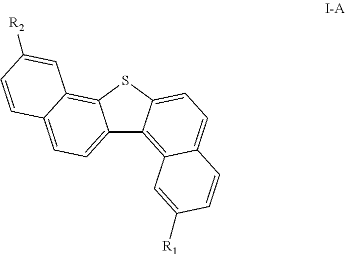
wherein R₁ is hydrogen, hydroxy, substituted or unsubstituted C₁-C₂₀ alkyl, or substituted or unsubstituted aryl; and R₂ is hydrogen, hydroxy, or substituted or unsubstituted C₁-C₂₀ alkyl.

2-9. (canceled)

10. A dinaphthothiophene compound of
(a) Formula I: 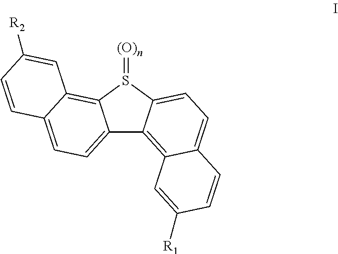
wherein R₁ is hydrogen, hydroxy, substituted or unsubstituted C₁-C₂₀ alkyl, or substituted or unsubstituted aryl; R₂ is hydrogen, hydroxy, or substituted or unsubstituted C₁-C₂₀ alkyl; and n is 0, 1, or 2;
(b) Formula II: 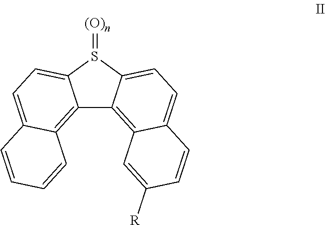
wherein R is hydrogen, hydroxy, substituted or unsubstituted C₁-C₂₀ alkyl, or substituted or unsubstituted aryl and n is 0, 1, or 2; or
(c) Formula III: 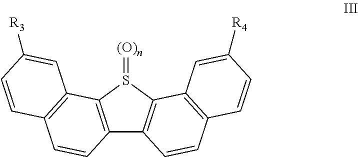
wherein R₃ is hydrogen hydroxy, substituted or unsubstituted C₁-C₂₀ alkyl, or substituted or unsubstituted aryl; R₄ is hydrogen, hydroxy, substituted or unsubstituted C₁-C₂₀ alkyl, or substituted or unsubstituted aryl; and n is 0, 1, or 2.

11. The compound of claim 10 wherein R₁ is hydrogen, hydroxy, C₁-C₂₀ alkyl, C₁-C₂₀ alkoxy, C₁-C₂₀ haloalkyl, C₁-C₂₀ haloalkoxy, aryl, alkyl-substituted aryl, halo-substituted aryl, or hydroxy-substituted aryl.

12. The compound of claim 10 wherein R₁ is hydrogen, hydroxy, C₁-C₁₀ alkyl, C₁-C₁₀ alkoxy, C₁-C₁₀ haloalkyl, C₁-C₁₀ haloalkoxy, aryl, alkyl-substituted aryl, halo-substituted aryl, or hydroxy-substituted aryl.

13. The compound of claim 10 wherein R₁ is hydrogen, hydroxy, methyl, ethyl, propyl, butyl, methoxy, ethoxy, propoxy, trifluoromethyl, phenyl, hydroxyphenyl, ethylphenyl, carboxyphenyl, naphthyl, anthracenyl, biphenyl, tolyl, cumyl, styryl, ortho-xylyl, meta-xylyl, para-xylyl, fluorophenyl, chlorophenyl, bromobenzyl, or iodobenzyl.

14. The compound of claim 10 wherein R₂ is hydrogen, hydroxy, C₁-C₂₀ alkyl, C₁-C₂₀ alkoxy, C₁-C₂₀ haloalkyl, C₁-C₂₀ haloalkoxy, aryl, alkyl-substituted aryl, halo-substituted aryl, or hydroxy-substituted aryl.

15. The compound of claim 10 wherein R₂ is hydrogen, hydroxy, C₁-C₁₀ alkyl, C₁-C₁₀ alkoxy, C₁-C₁₀ haloalkyl, C₁-C₁₀ haloalkoxy, aryl, alkyl-substituted aryl, halo-substituted aryl, or hydroxy-substituted aryl.

16. The compound of claim 10 wherein R₂ is hydrogen, hydroxy, methyl, ethyl, propyl, butyl, methoxy, ethoxy, propoxy, trifluoromethyl, phenyl, hydroxyphenyl, ethylphenyl, carboxyphenyl, naphthyl, anthracenyl, biphenyl, tolyl, cumyl, styryl, ortho-xylyl, meta-xylyl, para-xylyl, fluorophenyl, chlorophenyl, bromobenzyl, or iodobenzyl.

17. The compound of claim 10 wherein R₁ and R₂ are different.

18. A process for preparing a dinaphthothiophene compound, the process comprising:
reacting 2-bromthiophene with trans-2-(4-phenyl)vinylboronic acid in the presence of a catalyst comprising palladium, a base, and organic solvent to form (E)-2-styrylthiophene;
formylating (E)-2-styrylthiophene to form (E)-5-styrylthiophene-2-carbaldehyde;
reacting, in the presence of sodium hydride, (E)-5-styrylthiophene-2-carbaldehyde with a 4-substituted phosphonic acid diethyl ester compound of Formula B-1: 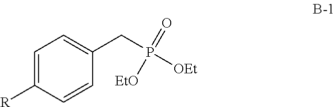
to form a compound of Formula B-2: 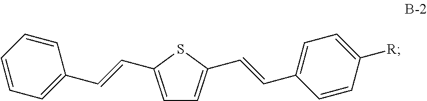
irradiating the compound of Formula B-2 with UV light in the presence of iodine and propylene oxide to form the dinaphthothiophene compound of Formula II-A: 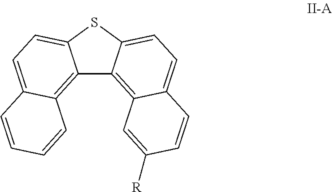
wherein R is hydrogen, hydroxy, substituted or unsubstituted C₁-C₂₀ alkyl, or substituted or unsubstituted aryl.

19-23. (canceled)

24. The compound of claim 10 wherein R is hydrogen, hydroxy, C₁-C₂₀ alkyl, C₁-C₂₀ alkoxy, C₁-C₂₀ haloalkyl, C₁-C₂₀ haloalkoxy, aryl, alkyl-substituted aryl, halo-substituted aryl, or hydroxy-substituted aryl.

25. The compound of claim 10 wherein R is hydrogen, hydroxy, C₁-C₁₀ alkyl, C₁-C₁₀ alkoxy, C₁-C₁₀ haloalkyl, C₁-C₁₀ haloalkoxy, aryl, alkyl-substituted aryl, halo-substituted aryl, or hydroxy-substituted aryl.

26. The compound of claim 10 wherein R is hydrogen, hydroxy, methyl, ethyl, propyl, butyl, methoxy, ethoxy, propoxy, trifluoromethyl, phenyl, hydroxyphenyl, ethylphenyl, carboxyphenyl, naphthyl, anthracenyl, biphenyl, tolyl, cumyl, styryl, ortho-xylyl, meta-xylyl, para-xylyl, fluorophenyl, chlorophenyl, bromobenzyl, or iodobenzyl.

27. A process for preparing a dinaphthothiophene compound, the process comprising:
formylating 3,4-dibromothiophene to form 4-bromothiophene-3-carbaldehyde;
reacting, in the presence of a catalyst comprising palladium, a base, and organic solvent, 4-bromothiophene-3-carbaldehyde with a phenylethenyl boronic compound of Formula C-1: 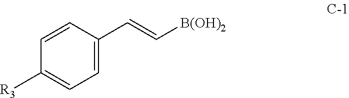
to form a 3-formyl-4-styrylthiophene compound of Formula C-2: 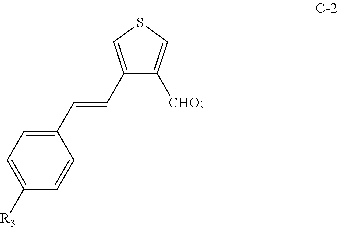
reacting, in the presence of sodium hydride, the 3-formyl-4-styrylthiophene compound of Formula C-2 with a 4-substituted phosphonic acid diethyl ester compound of Formula C-3: 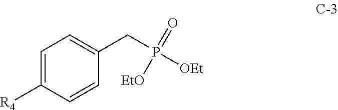
to form a compound of Formula C-4: 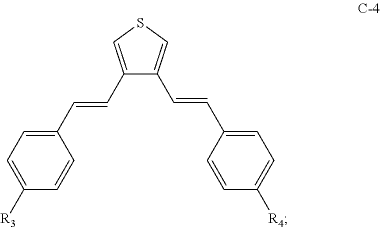
irradiating the compound of Formula C-4 with UV light in the presence of iodine and propylene oxide to form the dinaphthothiophene compound of Formula III-A: 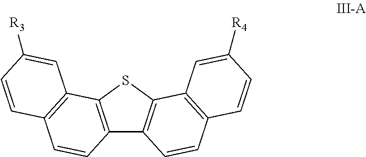
wherein R₃ is hydrogen, hydroxy, substituted or unsubstituted C₁-C₂₀ alkyl, or substituted or unsubstituted aryl and R₄ is hydrogen, hydroxy, substituted or unsubstituted C₁-C₂₀ alkyl, or substituted or unsubstituted aryl.

28-36. (canceled)

37. The compound of claim 10 wherein R₃ is hydrogen, hydroxy, C₁-C₂₀ alkyl, C₁-C₂₀ alkoxy, C₁-C₂₀ haloalkyl, C₁-C₂₀ haloalkoxy, aryl, alkyl-substituted aryl, halo-substituted aryl, or hydroxy-substituted aryl.

38. The compound of claim 10 wherein R₃ is hydrogen, hydroxy, C₁-C₁₀ alkyl, C₁-C₁₀ alkoxy, C₁-C₁₀ haloalkyl, C₁-C₁₀ haloalkoxy, aryl, alkyl-substituted aryl, halo-substituted aryl, or hydroxy-substituted aryl.

39. The compound of claim 10 wherein R₃ is hydrogen, hydroxy, methyl, ethyl, propyl, butyl, methoxy, ethoxy, propoxy, trifluoromethyl, phenyl, hydroxyphenyl, ethylphenyl, carboxyphenyl, naphthyl, anthracenyl, biphenyl, tolyl, cumyl, styryl, ortho-xylyl, meta-xylyl, para-xylyl, fluorophenyl, chlorophenyl, bromobenzyl, or iodobenzyl.

40. The compound of claim 10 wherein R₄ is hydrogen, hydroxy, C₁-C₂₀ alkyl, C₁-C₂₀ alkoxy, C₁-C₂₀ haloalkyl, C₁-C₂₀ haloalkoxy, aryl, alkyl-substituted aryl, halo-substituted aryl, or hydroxy-substituted aryl.

41. The compound of claim 10 wherein R₄ is hydrogen, hydroxy, C₁-C₁₀ alkyl, C₁-C₁₀ alkoxy, C₁-C₁₀ haloalkyl, C₁-C₁₀ haloalkoxy, aryl, alkyl-substituted aryl, halo-substituted aryl, or hydroxy-substituted aryl.

42. The compound of claim 10 wherein R₄ is hydrogen, hydroxy, methyl, ethyl, propyl, butyl, methoxy, ethoxy, propoxy, trifluoromethyl, phenyl, hydroxyphenyl, ethylphenyl, carboxyphenyl, naphthyl, anthracenyl, biphenyl, tolyl, cumyl, styryl, ortho-xylyl, meta-xylyl, para-xylyl, fluorophenyl, chlorophenyl, bromobenzyl, or iodobenzyl.

43. (canceled)
